(19) United States
(12) Patent Application Publication
(10) Pub. No.: US 2020/0237836 A1
(43) Pub. Date: Jul. 30, 2020

(54) COMPOSITIONS THAT METABOLIZE OR SEQUESTER FREE SUGAR MONOMERS AND USES THEREOF

(71) Applicant: Evolve BioSystems, Inc., Davis, CA (US)

(72) Inventors: David KYLE, Davis, CA (US); David Mills, Davis, CA (US); Carlito Lebrilla, Davis, CA (US); Samara Freeman-sharkey, Davis, CA (US); Steven Frese, Davis, CA (US)

(21) Appl. No.: 16/676,163

(22) Filed: Nov. 6, 2019

Related U.S. Application Data
(63) Continuation of application No. 15/558,110, filed on Sep. 13, 2017, now abandoned.
(60) Provisional application No. 62/133,239, filed on Mar. 13, 2015.

(51) Int. Cl. A61K 35/744; A61K 31/7012; A61K 35/745; A61K 35/747; A23L 33/00; A23K 10/18; A61K 35/74; A23K 50/20; A61K 31/7004; A61K 9/16; A23L 33/125; A23L 33/135; A23K 50/60; A23K 50/30; A61K 9/19; A23L 33/21

(52) U.S. Cl. CPC A61K 35/744; A23L 33/21; A61K 35/745; A61K 35/747; A23L 33/40; A23K 10/18; A61K 35/74; A23K 50/20; A61K 31/7004; A61K 9/1635; A23L 33/125; A23L 33/135; A23K 50/60; A23K 50/30; A61K 9/19; A61K 9/1623; A61K 31/7012

(57) ABSTRACT

Compositions comprising at least two non-pathogenic microbes are described herein. The non-pathogenic microbes may be from a first species capable of internalizing and/or metabolizing dietary glycans and/or from a second species capable of consuming and metabolizing free sugar monomers. Methods of making and use in treating and/or preventing the overgrowth of pathogenic bacteria in mammals are also described herein.

### FIELD OF THE INVENTION

The embodiments described herein relate generally to promoting health in a mammal, and more particularly, to modulating the microbiome of individual humans. Further, the embodiments relate to methods of treating and/or preventing the overgrowth of pathogenic bacteria in mammals.

### BACKGROUND

The intestinal microbiome is the community of microorganisms that live within the gastrointestinal tract, the majority of which is found in the large intestine or colon. In a healthy individual, most dietary nutrients that are consumed are absorbed by the body before they reach the colon. Many foods, however, contain indigestible carbohydrates (i.e dietary fiber) that remain intact and are not absorbed during transit through the gut to the colon. The colonic microbiome is rich in bacterial species that are able to partially consume these fibers and utilize the constituent sugars for energy and metabolism. Methods for measuring dietary fiber in various foods are well known to one of ordinary skill in the art.

In mammalian species, the nursing infant's intestinal microbiome is quite different from that of an adult microbiome in that the adult gut microbiome generally contains a large diversity of organisms all present and a low percentage of the total population. The nursing infant's microbiome, on the other hand can be made up almost exclusively (up to 80%) of a single species.

The transition from the simple, non-diverse microbiome of the nursing infant to a complex, diverse microbiome of an adult reflects the mammal's transition from a single nutrient source of a rather complex fiber (e.g, maternal milk oligosaccharides) to more diverse dietary fiber sources that are less complex.

Dysbiosis, is a term for a microbiome that is discordant relative to the natural healthy microbial population. An example of a natural state of a mammalian microbiome throughout evolution is that of the gastrointestinal tract of healthy human infants that are vaginally delivered (i.e., inoculated with specific microbes from maternal sources), and breast fed. There may be various reasons for dysbiosis in human infants including surgical delivery via Cesarean Section, use of alternative foods or formulas (rather than nursing), the extensive use of antibiotics, sanitation practices in neonatal facilities/settings, and the microbial environments of homes and hospitals where that infant is raised.

Dysbiosis can also occur in humans of all age groups, and in other domesticated mammalian species such as, but not limited to, agriculturally-relevant mammals (e.g., cows, pigs, rabbits, goats, and sheep), mammalian companion animals (e.g., cats, dogs, and horses), and performance mammals (e.g., thoroughbred race horses, racing camels, and working dogs) for similar reasons of hygiene, extensive use of antibiotics, and the industrialization of foods and feeds for those humans and animals.

Previous treatment protocols for a dysbiotic mammal include the administration of an antibiotic that eradicated all, or the majority of, bacteria in the microbiome. For example, Necrotizing Enterocolitis (NEC), a condition that occasionally develops in very small preterm infants, is a severe condition which often requires major surgery to resect certain parts of the necrotic bowel having life-long sequelae, and can often lead to the death of the infant, is universally treated with antibiotics.

Other dysbiotic gut microbial community compositions can exist within adult or young mammals (e.g., piglets, foals, and calves). Under intensive agricultural production of pigs and horses, antibiotics are frequently used prophylactically, and the microbial diversity of the animals under these conditions is lowered and a dysbiotic gut microbial community ensues. Ironically, this can often lead to pathology (e.g., scours in piglets, or outbreaks of pathogenic bacteria such as Clostridium difficile or C. perfringens in foals) that are treated by yet more powerful antibiotics to prevent the life threatening diarrhea and possibly death. Presently, the only choice for the elimination of these pathogenic bacteria in such situations is the continued and extensive use of antibiotics and supportive or palliative care. Thus, there is a need for an effective method to reduce dysbiosis and prevent disease in mammals of all ages (including humans as well as companion, performance, and production animals) that does not involve the additional administration of antibiotics.

### SUMMARY

The instant invention relates in part to the inventors' discovery that mammalian milks, and especially the glycan components of milk, have evolved to feed two consumers: the immediate offspring; and the offspring's appropriate gut bacteria. The inventors have discovered that in the absence of the evolutionary-associated bacteria (or the presence of a dysbiotic gut), the indigestible glycans of mammalian milk become susceptible to hydrolysis by other bacteria. This releases Free Sugar Monomers (FSMs), which are capable of enabling the growth of opportunistic or highly destructive pathogens that would not have flourished otherwise. The term “dietary glycans”, as used herein, refers to those indigestible glycans, sometimes referred to as “dietary fiber”, or the carbohydrate polymers which are not hydrolyzed by the endogenous enzymes in the digestive tract (e.g., the small intestine) of the mammal.

Some embodiments of the invention involve compositions and methods of delivering to the gut of a dysbiotic mammal, compositions that include components capable of consuming the dietary glycans. Such compositions can reduce the concentration of FSMs in the mammal. The reduction of FSMs and dietary glycans can minimize the likelihood of an overpopulation of pathogenic bacteria that can harm that mammal. In some embodiments the compositions comprise certain bacteria (alive or dead) or other orally provided compounds that bind and/or metabolize the FSMs, thereby preventing them from being used as an energy source by the pathogenic bacteria.

Some of the embodiments of the present invention provide diagnostics for the presence of substrates enabling growth of pathogenic bacteria within mammalian neonates. Specifically, some embodiments provide diagnostics to determine the presence of FSMs.

Some of the embodiments of the present invention deliver a suite of a) microorganisms (e.g., bacteria or yeast) that act as probiotics to actively remove substrates including intact dietary glycans and FSMs; b) enzymes capable of inactivating or eliminating dietary glycans and/or sugars; and/or c) binding agents that physically bind and render free sugars monomers unavailable as substrates supporting the growth of pathogenic microorganisms.

Some embodiments of the invention provide a composition administered to reduce the concentration of FSMs that may be the consequence of the use of antibiotics to treat the pathogenic bacterial overgrowth in mammals including humans.

### BRIEF DESCRIPTION OF THE DRAWINGS

FIG. 1: Graph showing typical piglet E. coli isolate on pig milk sugar constituent sugars vs. conjugated glycans.

FIG. 2: stacked bar chart showing relative abundance of populations of Bacteroidaceae (in yellow), and in blue, the Enterobacteriaceae are strongly correlated (r2=0.661, p<0.001) in the feces of young pigs. Communities in weaned animals are boxed.

FIG. 3A: Chart showing taxonomic identity of metagenomic reads annotated as sialidase enzyme.

FIG. 3B: Chart showing significant sialidase relative abundance differences between milk and weaning diets.

FIG. 4: Chart showing free sialic acid concentration in the feces of nursing and weaned piglets.

FIG. 5: Chart showing average Enterobacteriaceae populations over time in pigs (left axis, bars, nursing, blue; weaned, red), are significantly different (p<0.001) between diets as well as concentrations of free sialic acid, p<0.001 (Right axis, whiskers, nursing, blue; weaned, red).

FIG. 6: Chart showing biogeographical relative abundances of Bacteroidales and Enterobacteriaes in the gut of 14 day old nursing pigs.

FIG. 7: Graph showing treating 14 d old pigs by gavage with Lactobacillus UCD14261 led to significant reductions in Enterobacteriaceae populations.

FIG. 8: Chart showing distinct differences in “At risk” or high-Enterobacteriaceae versus “NR” “No risk” pigs prior to gavage with Lactobacillus.

FIG. 9: Chart showing “at risk” (AR) or high-Enterobacteriaceae pigs could be rescued by gavage with Lactobacillus to resemble No Risk or Non Responder animals. Letters denote significance groups (a, b; p<0.05).

FIG. 10: Model for PMO Consumption.

### DETAILED DESCRIPTION OF PREFERRED EMBODIMENTS

Dysbiosis in a mammal, especially an infant mammal, can be observed by the physical symptoms of the mammal (e.g., diarrhea, digestive discomfort, inflammation, etc.) and/or by observation of the presence of FSMs in the feces of the mammal. Additionally, the infant mammal may have an increased likelihood of becoming dysbiotic based on the circumstances in the environment surrounding the mammal (e.g., an outbreak of disease in the surroundings of the mammal, formula feeding, cesarean birth, etc.).

Most gut microbes living in a medium comprising complex glycans will secrete hydrolytic enzymes into their surrounding environment to cleave off digestible fragments (FSM) that can be consumed by those microbes for energy and/or other needs of those microbes. The inventors have discovered that some microbes can grow exclusively on the complex glycans found in mammalian milk by first internalizing many of those complex glycans (milk oligosaccharides) with limited prior hydrolysis or without prior hydrolysis altogether. The internalized glycans are hydrolyzed in such a way that the resulting FSMs are released in the cell cytoplasm, and can be metabolized for energy and/or other needs of those microbes without their release into the external environment. These latter microbes typically do not secrete carbohydrate-active hydrolases. Microbes that secrete carbohydrate-active hydrolases frequently leave significant quantities of residual fragments or FSMs in the surrounding medium, whereas microbes that evolved to consume oligosaccharides from mammalian milk by internalization do not leave residual FSMs in the surrounding medium. Such circumstances occur when these respective organisms are growing in the intestines of mammals.

Thus, the infant mammal for which treatment and/or prevention of certain conditions is prescribed using the present invention can be one that: (a) has a physical symptom indicative of dysbiosis (e.g., diarrhea or digestive discomfort); (b) has a measurable level of FSMs in their feces; and/or (c) has an increased likelihood of becoming dysbiotic based on the environmental conditions surrounding the mammal (e.g., an outbreak of disease in the surroundings of the mammal, formula feeding, cesarean birth, etc.). The mammal may be a human, a cow, a pig, a rabbit, a goat, a sheep, a cat, a dog, a horse, or a camel.

Levels of FSMs in the feces of the infant mammal increase when the bacteria making up the gut microbiome are not able to completely consume the dietary glycans. As a result of the partial extracellular degradation of the dietary glycans there is an elevation of FSMs and disaccharides in the lower bowel. The FSMs can include, but are not limited to, fucose, sialic acid, N-acetylglucosamine, glucose, gluconate, mannose, N-acetylgalactosamine, ribose, and/or galactose.

Certain pathologies in mammals, including, but not limited to humans, horses, and pigs, cows, rabbits, goats, sheep, dogs, horses, camels, or cats, are correlated with the overgrowth of certain pathogenic bacteria in the gut such as, but not limited to, Proteobacteria, including Enterobacteriaceae, and Firmicutes, including Clostridium. The inventors have observed that the overgrowth (a bloom) of such problematic bacteria appears to be correlated with the abundance of FSMs produced by the partial digestion of dietary glycans. The inventors have also determined that the root cause of pathogenesis as a result of dysbiosis in the gut is related to the presence in the lower bowel of excess FSMs including, but not limited to, fucose, sialic acid, N-acetylglucosamine, N-acetylgalactosamine, and gluconate. An excess of FSMs can be due to an incomplete digestion of dietary glycans (such as those found in mammalian milk and other food sources) by the resident gut microbiome. Thus, the association of FSMs and gut pathogens is causal and problematic.

In the case of humans, especially in Western countries where the population has easy access to modern medical care and practices, there are high rates of infants are born by Caesarean Section (C-Section), high rates of usage of artificial milk (infant formula) early in life, and high rates of treatment with antibiotics at an early stage, or during the mother's life. In all of these cases, the human infants can quickly develop a gastrointestinal microbiota that is profoundly different than that of an ‘ancestral’ or ‘ideal’ vaginally-delivered, breast-fed baby. The microbiome of a normal adult human is highly complex relative to that of the breast fed infant. The vaginally-delivered, breast fed infant, for example, has a microbiome that, after an initial stage of colonization, is ideally dominated by a single genus of bacteria (Bifidobacterium) and often by a single species and subspecies (Bifidobacterium longum subsp. infantis (B. infantis)). This milk-guided, B. infantis-dominated microbiome typically changes to a complex adult-like microbiome quite rapidly following the cessation of the consumption of human milk by the infant. The microbiome change resulting from this change in the infant's diet is quite different from the microbiome change found following antibiotic treatment of a human infant, child or adult, or any other mammal, where the microbiome becomes profoundly disrupted or dysbiotic.

The infant mammals of the present invention may have been treated with antibiotics, or may be contemporaneously treated with antibiotics, or may have been born to animals treated with antibiotics or may be born to animals contemporaneously treated with antibiotics. The infant mammal may be a human, a cow, a pig, a rabbit, a goat, a sheep, a cat, a dog, a horse, or a camel that has been, or is being, treated with antibiotics.

In some embodiments, the invention provides a composition which comprises at least two non-pathogenic microbes. When used herein, the term “non-pathogenic microbes” means microbes that are unable to cause a disease and may also be called “commensal microbes” which means living together without causing harm to each other. One of the non-pathogenic microbes can be from a first species (e.g., a yeast or a bacteria) which is capable of internalizing, hydrolyzing, and/or metabolizing dietary glycans. The first species can be a Bifidobacterium. The bifidobacteria may be B. longum (for example B. longum subsp. infantis, B. longum subsp. longum), B. breve, or B. pseudocatenulatum.

In some embodiments, the first species is B. longum subsp. infantis. The B. infantis may be activated. Activation of B. infantis is described in PCT/US2015/057226, the disclosure of which is incorporated herein in its entirety.

In some embodiments, the second non-pathogenic microbe is from a second species (e.g., a yeast or bacteria) which is capable of consuming and metabolizing at least one type of FSM. In some embodiments, the second species is a Pediococcus, Lactobacillus, or bifidobacteria. In some embodiments, the second species can be, but is not limited to, B. infantis, B. breve, B. bifidum, B. longum, B. adolescentis, B. animalis, P. pentosaceus, P. stilesii, P. acidilacti, P. argentenicus, P. claussenii, L. reuteri, L. acidophilus, L. planatarum, L. casei, L. rhamnosus, L. brevis, L. fermentum, L. crispatus, L. johnsonii, L. gasseri, L. mucosae, and/or L. salivarius.

In some embodiments, the second species is selected due to the cause of the actual or potential dysbiosis of the infant mammal and the second species' preference for consumption of the FSM underlying the actual or potential dysbiosis. For example, the second species may be selected based on the ability of the microbe's preference for FSM consumption (described in Table 1 below). While the microbe may be capable of consuming and metabolizing the FSM, the microbe may not prefer to consume the FSM unless no other food source is available.

TABLE 1
Listing of common intestinal nnicrobiota and preferences for free sugar consumption

| | Monomers | | | | | Dimers/Trimers | | | |
|---|---|---|---|---|---|---|---|---|---|
| Organism | Fucose | Sialic Acid | N-acetylglucosamine | Glucose | Galactose | Lactose | Sialyllactose | Fucosyllactose | Lacto-N-Biose |

Bifidobacteria

| | | | | | | | | | |
|---|---|---|---|---|---|---|---|---|---|
| B. infantis | * | + | + | + | + | + | + | + | + |
| B. breve | * | + | + | + | + | + | + | + | + |
| B. bifidum | * | − | + | + | + | + | + | + | + |
| B. longum | * | − | + | + | + | + | − | − | + |
| B. adolescentis | − | − | + | + | + | + | − | − | − |
| B. animalis | − | − | − | + | + | + | − | − | − |

Lactobacilli

| | | | | | | | | | |
|---|---|---|---|---|---|---|---|---|---|
| L. reuteri | − | − | + | + | + | + | − | − | − |
| L. acidophilus | − | − | + | + | + | + | − | − | − |
| L. plantarum | − | + | + | + | + | + | − | − | − |
| L. casei | − | − | + | + | + | + | − | + | + |
| L. rhamnosus | − | − | + | + | + | + | − | + | |
| L. brevis | − | − | + | + | + | + | + | − | − |
| L. fermentum | − | − | − | + | + | + | − | − | − |
| L. crispatus | − | + | + | + | + | + | − | − | − |
| L. johnsonii | − | − | + | + | + | + | − | − | − |
| L. gasseri | − | + | + | + | + | + | − | − | − |
| L. mucosae | − | − | − | + | + | + | − | − | − |
| L. salivarius | − | + | + | + | + | + | + | − | − |

Pediococcus

| | | | | | | | | | |
|---|---|---|---|---|---|---|---|---|---|
| P. stilesii | + | ND | + | + | + | + | ND | ND | ND |
| P. pentosaceus | + | ND | + | + | + | + | ND | ND | ND |
| P. acidilacti | − | ND | + | + | + | − | ND | ND | ND |
| P. argentinicus | − | ND | + | + | + | − | ND | ND | ND |

* Predicted but not observed
ND = Not Determined

In some embodiments, the FSM underlying the actual or potential dysbiosis is identified by measuring the FSMs present in a fecal sample of the infant mammal, or by examining the complex glycans in the animal's diet. The second species can be then selected for its preference to consume the FSMs measured in the fecal sample of the infant mammal. The infant mammal can be determined to have FSM in the feces in an amount of at least 1 ug, at least 5 ug, at least 10 ug, at least 15 ug, at least 20 ug, at least 25 ug, at least 50 ug, at least 75 ug, at least 100 ug of FSM (e.g., N-acetylglucosamine, fucose, or sialic acid) per gram dry weight of feces of the infant mammal.

In some embodiments, the FSM underlying the actual or potential dysbiosis is identified by identifying the pathogenic microbe and the preferred free sugar consumption of the pathogenic microbe. For example, it is known that Clostridium difficile consumes sialic acid. Thus, if an infant mammal is susceptible to and/or exposed to, for example, an environment enriched in C. difficile, the second species could be selected for its preference to consume sialic acid.

The composition can comprise a first species of a non-pathogenic microbe that is present in an amount of about 5 to about 95% of the total of non-pathogenic microbes. For example, the first species can be present in an amount of 5%, 10%, 15%, 20%, 25%, 30%, 35%, 40%, 45%, 50%, 55%, 60%, 65%, 70%, 75%, 80%, 85%, 90%, or 95% (e.g., about 10% to about 90%, or about 20% to about 80%) of the total amount of non-pathogenic microbes. The composition can comprise a second species of a non-pathogenic microbe that is present in an amount of about 5 to about 95% of the total of non-pathogenic microbes. For example, the second species can be present in an amount of 5%, 10%, 15%, 20%, 25%, 30%, 35%, 40%, 45%, 50%, 55%, 60%, 65%, 70%, 75%, 80%, 85%, 90%, or 95% (e.g., about 10% to about 90%, or about 20% to about 80%) of the total amount of non-pathogenic microbes. In some embodiments, the total number amount of non-pathogenic microbes is 1 billion to about 10 million to about 500 billion cfu per gram dry weight of the composition.

Any of the compositions described herein can be in the form of a dry powder, a dry powder suspended in an oil, or a liquid suspension of a culture of the bacteria. The composition can comprise a total count of live bacteria from about 10 million to about 500 billion cfu per gram dry weight. The dry powder can be freeze-dried or spray dried. The freeze-dried compositions are preferably frozen in the presence of a suitable cryoprotectant. The cryoprotectant can be, for example, glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, or taurine. The composition may also comprise from about 5 to 90% of dietary glycans from a mammalian source including, but not limited to a human, swine, or bovine species.

In an embodiment, the composition is capable of growing on dietary glycans wherein less than 20% of the sialic acid content and 20% of the fucose content of the dietary glycans remains as FSMs after a culture of the composition has ceased to grow. In another embodiment, the composition is capable of growing on dietary glycans wherein less than 10% of the sialic acid content and 10% of the fucose content of the dietary glycans remains as FSMs after a culture of the composition has ceased to grow. In a preferable embodiment the composition is capable of growing on dietary glycans wherein less than 5% of the sialic acid and 5% of the fucose of the milk oligosaccharides remains as FSMs after a culture of the composition has ceased to grow. In a most preferable embodiment the composition is capable of growing on dietary glycans wherein less than 1% of the sialic acid and 1% of the fucose of the milk oligosaccharides remains as FSMs after a culture of the composition has ceased to grow.

In some embodiments, the first species of non-pathogenic microbes contains a gene coding for a sialidase or a fucosidase, and the second species of non-pathogenic microbes contains a gene coding for a sialic acid or a fucose transporter. In another embodiment one of the species contains a gene coding for a complex oligosaccharide transporter. In some embodiments, one of the live bacterial species is Bifidobacterium longum and in a most preferred embodiment, one or both of the live bacterial species is Bifidobacterium longum subspecies infantis.

In various embodiments of the invention, one or both of the bacterial species may be rendered nonviable by any of a number of treatments including, but not limited to, heating, freezing sonication, osmotic shock, low pH, high pH, or gluteraldehyde treatment. Under such conditions the dietary glycans and/or FSM binding proteins on the surface of the cell are still intact and the nonviable bacterial cell can bind but not metabolize the dietary glycans/sugar.

In various embodiments, the gene or genes for a FSM transporter such as but not limited to the sialic acid or fucose transporters, or dietary glycans binding protein, can be expressed in a recombinant cell which can be provided in a viable or nonviable fashion to a subject in need of lowering their fecal FSM levels. In another embodiment, certain genes responsible for the uptake of the FSMs could also be overexpressed in another bacterial or yeast strain to further enhance that organism's ability to consume any residual FSMs in the lower colon of a mammalian species. In yet another embodiment, genes for specific dietary glycan binding molecules (e.g., certain cell surface lectins or selectins chosen for the binding of FSMs, preferably sialic acid and fucose) may also be incorporated into a recombinant organism to sequester FSMs and prevent the pathogenic bacteria from utilizing FSMs as an energy source. Additionally, specific non-protein sugar binding molecules such as but not limited to cyclodextrins, dextran sulphates, etc., can also be used in the composition for sequestration of FSMs.

In some embodiments, additional biological sources such as any other non-pathogenic bacteria capable to taking up residual FSMs can be included. Such organisms can be obtained by screening for growth on FSMs such as, but not limited to, N-acetylglucosamine, fucose, gluconate and sialic acid or combinations of these sugars. Such organisms can be obtained by first mutagenizing nonpathogenic strains of bacteria by standard procedures known in the art such as, but not limited to, UV mutagenesis and chemical mutagenesis, and using the individual sugars as a positive selection procedure to identify mutant strains that are constitutively active in terms of uptake and metabolism of such FSMs.

In additional embodiments, any of the compositions described herein are provided orally with or without packaging in a slow release formulation. The slow release formulation can be formulated so that the composition will successfully transit the low pH of the stomach and other digestive enzymes and detergents in the upper small intestine in order to provide an effective delivery of the dietary glycan-binding molecules to the large intestine. Alternatively, these materials can be provided anally through the use of such means as, but not limited to, a suppository, an enema, or a douche, directly into the colon in a fashion similar to a fecal transplant.

In various embodiments, the health of a dysbiotic mammal can be improved by administering to the mammal any of the compositions described herein. The mammal can be determined to have FSMs in the feces in an amount of at least 1 ug, at least 5 ug, at least 10 ug, at least 15 ug, at least 20 ug, at least 25 ug, at least 50 ug, at least 75 ug, at least 100 ug of FSM (e.g., N-acetylglucosamine, fucose, or sialic acid) per gram dry weight of feces of the mammal. The mammal can be administered any of the compositions described herein. The mammal can be administered a composition comprising non-pathogenic microbes comprising live bacteria that is capable of metabolizing or sequestering the FSMs in an amount of from about 10 million to about 500 billion cfu per gram.

In some embodiments, the presence of FSM in an infant mammal's feces or the composition of complex glycans in the infant mammal's diet are determined and the presence is reported. A recommendation of administering a composition based on the presence of the FSMs or possible FSMs (constituents of the dietary glycans) can be made. Any of the compositions described herein can be recommended to be administered to the infant mammal. The composition can comprise non-pathogenic microbes that are capable of metabolizing or sequestering the FSMs. The composition can be subsequently administered to the mammal to treat the mammal. The infant mammal being treated can be, but is not limited to, a human, a cow, a pig, a rabbit, a goat, a sheep, a cat, a dog, a horse, or a camel.

An assessment can be made for the presence of FSMs in the feces of the infant mammal. Individuals having the presence of such FSMs in the feces at levels of from 1 ug to 100 mg/g dry weight of feces will be candidates for treatment using the compositions of the instant invention. In a preferred embodiment the levels of fecal FSMs would be from 5 ug to 50 mg/g dry weight of feces, and in a most preferred embodiment the level of fecal FSMs would be from 5 ug to 5 mg/g dry weight of feces.

An embodiment of the instant invention may include the following steps; 1) a subject suitable for the treatment by this invention is identified by the presence of FSMs in the feces at levels of at least 5 ug/g dry weight of feces, or some other form of intestinal distress; 2) a composition that will sequester and/or consume FSMs is prepared; and 3) the FSM-sequestering and/or -consuming composition is provided to the subject in need of reducing the levels of FSMs.

### EXAMPLES

### Example 1. Determination of a Mammalian Subject Predisposed to Pathogenic Bacterial Blooms

A routine sample of the subject's feces is analyzed by standard processes well known in the art (see, e.g., Le Parc et al., “Rapid Quantification of Functional Carbohydrates in Food Products”, Food and Nutrition Sciences (2014), Vol. 5, pp. 71-78), for the presence of N-acetyl glucosamine, sialic acid, gluconate and/or fucose. If the determination of the analysis indicates the presence of any of the FSMs at levels in excess of 5 ug/g dry weight of feces, then the subject is a candidate for treatment.

### Example 2. Preparation of the FSM-Sequestering Composition

A sample of B. infantis is isolated by the cultivation of the feces of a vaginally-delivered and breast-fed human infant on a medium that contains human milk oligosaccharides (HMOs) as a sole source of energy for the growth of the organism. Alternatively, a strain of B. infantis can be obtained from a commercial culture collection such as The American Type Culture Collection (ATCC) of Manassas, Va. A species of Bifidobacterium, Pediococcus, or Lactobacillus that can consume N-acetylglucosamine, sialic acid, gluconate or fucose such as, but not limited to, B. longum, B. breve, B. pseudocatenulatum, B. dentium, P. pentosaceus, P. stilesii, P. acidilacti, P. argentinicus, L. reuteri L. plantarum, L. pentosus, L. salivarius, L. crispatus, L. coleohominis, L. antri, L. sakei and L. casei is used in conjunction with the B. infantis. Pure cultures of both organisms are grown independently using conventional commercial fermentation techniques in fermenter of greater than 500 L in volume, and the growth medium may include mammalian milk complex dietary glycans and/or FSMs as a component of the carbon source. Each of the cell broths are concentrated by centrifugation and blended separately with a cryopreservative component, such as but not limited to trehalose, prior to freezing and subsequent drying by reduced atmospheric pressure (i.e., freeze drying). Once dried the two pure cultures are blended in a ratio of from 1:5 to 5:1.

### Example 3. Treatment of a Subject in Need of Supplementation Using the Composition

A human infant with a fecal FSM concentration of greater than 5 ug/g dry weight (gdw) feces is selected for supplementation with the composition of this invention. Mixtures are produced comprising from 10 million to 100 billion cfu/gdw of B. infantis and 10 million to 100 billion cfu/gdw of Lactobacillus sp. Such a composition is provided at a dosage of from 10 million to 100 billion cfu/gdw/day of combined Bifidobacterium and Lactobacillus. Such mixtures are provided to the infant in need of supplementation for a period of at least 5 days.

### Example 4

Newborn foals born to mares at a large horse breeding barn were monitored during an outbreak of severe hemorrhagic diarrhea among the foals. The foals were found to be culture- and toxin-positive for Clostridium difficile. Seventeen foals were born during the initial stage of the outbreak, of which fifteen animals became ill and required intervention, according to the standard of care as described in the Merck Veterinary Manual. Standard of care involved metronidazole treatment given at a dose of 15-20 mg/kg, PO, tid-qid. And may also involve administration of large volumes of interveneous polyionic fluids, with supplemental electrolytes (potassium, magnesium, and calcium), plasma or synthetic colloids for low oncotic pressure, anti-inflammatories such as flunixin meglumine, and broad-spectrum antibiotics if the horse is leukopenic and at risk of bacterial translocation across the compromised GI tract.

Of these seventeen, fifteen developed loose stool or diarrhea lasting 3-4 days, and 2 died as a result of the infection. After observing the outbreak, the care regimen was changed such that newly delivered foals were provided a formulation of 3×10¹² CFU Bifidobacterium longum EVBL001 and 5×10⁹ CFU of Lactobacillus plantarum EVLP001 every 12 hours, starting 12 hours after birth. The two foals that were provided with the formulation at 12 hours of age still developed diarrhea, but recovered within 8 hours compared to 3-4 days with standard of care. The care regimen was changed to dose these animals with EVBL001 and EVLP001 at birth and every twelve hours thereafter. None of the foals provided with this dose starting at birth developed diarrhea (n=6).

Recovery time for the two treated animals that eventually developed the infection was approximately eight hours, which was significantly shorter than the normal recovery time of at least 3-4 days for animals given the standard care regimen. No adverse events were recorded among the treated animals and the dosages were well tolerated. A Fisher's exact test of the two populations (Standard of Care and Probiotic treated) yields a significant difference in incidences of C. difficile infection (p=0.0016) (Table 1).

TABLE 1
A 2 × 2 Contingency table analyzed by Fisher's
Exact test indicates a significant reduction in sick
animals among those treated with the probiotic mixture
(Treated), relative to the standard of care (Control).

| | Healthy | Diarrhea | Total |
|---|---|---|---|
| Control | 2 | 15 | 17 |
| Treated | 6 | 2 | 8 |
| Total | 9 | 17 | 26 |

Fisher's Exact Test
The two-tailed P value equals 0.0036

Two treatment options were attempted. In the first, animals were dosed at 12 hours of life, but this fails to significantly reduce incidence of diarrhea (given the small n), though the severity (duration) was dramatically shortened to 12 hours or less (p=0.0074; Fisher exact test, comparing populations of diarrheal foals segregated by duration of diarrhea). The second option, dosing at birth, was significant at reducing incidences of diarrhea (p=0.0025). All animals were dosed at birth with 6.6 mg/kg of ceftiofur (Excede), and this did not affect health outcome, related to diarrhea. Additionally, the treated population did not develop foal heat diarrhea, which typically affects >50% of animals, and requires treatment in approximately 10% of cases (Weese and Rousseau 2005). If a >50% risk is extrapolated to a hypothetical population of 8 animals to match the 8 observed; this yields a significant reduction in foal heat diarrhea (p=0.0256).

The results described above demonstrate that administration of a composition that includes Bifidobacteria (e.g, B. longum subspecies infantis) with a Lactobacillus (e.g., L. plantarum) that was chosen to consume the FSMs that a known pathogen (e.g., a Clostridium species) preferred to consume, was effective at reducing the dysbiotic episodes and subsequent life-threatening diarrhea for the newborn foals. This example is not limited to newborn foals, but demonstrates that administration of the compositions described herein can be effective to reduce or eliminate dysbiotic episodes in mammals.

### Example 5

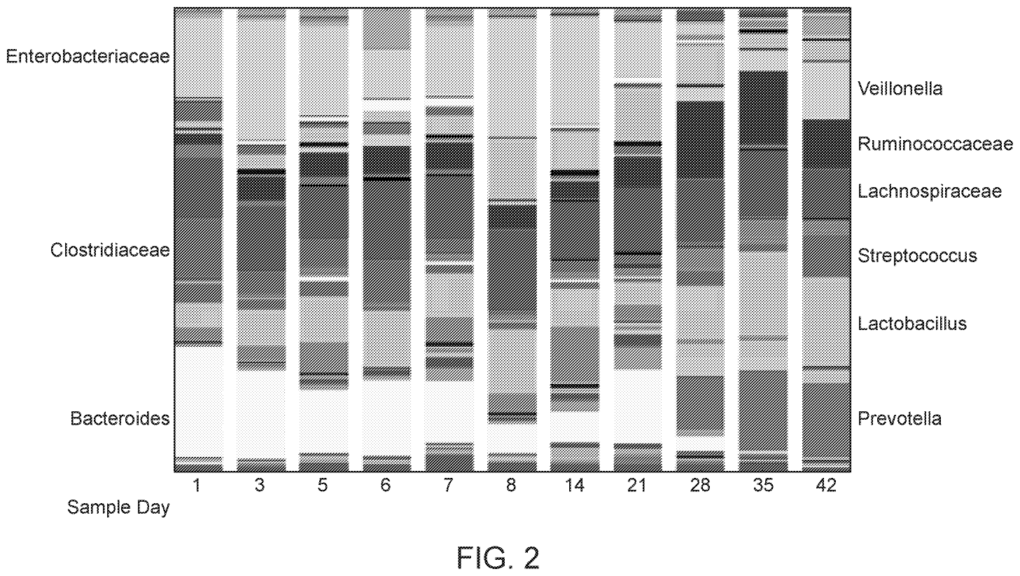

To understand the relationship of the gut microbiota with pig dietary glycans, an experiment was conducted to monitor the temporal changes in the fecal microbiota of pigs from birth through weaning. Fecal microbial populations remained stable while the animals were nursing, but changed dramatically at weaning, when dietary glycans were removed from the diet. The dominant taxonomic changes that were found during this transition were in the families Enterobacteriaceae (which includes E. coli) and the Bacteroidaceae (which includes a genus common to the gut microbiota, Bacteroides) (FIG. 2). FIG. 2 is a stacked bar chart showing relative abundance of populations of Bacteroidaceae (in yellow), and in blue, the Enterobacteriaceae are strongly correlated (r2=0.661, p<0.001) in the feces of young pigs. Communities in weaned animals are boxed.

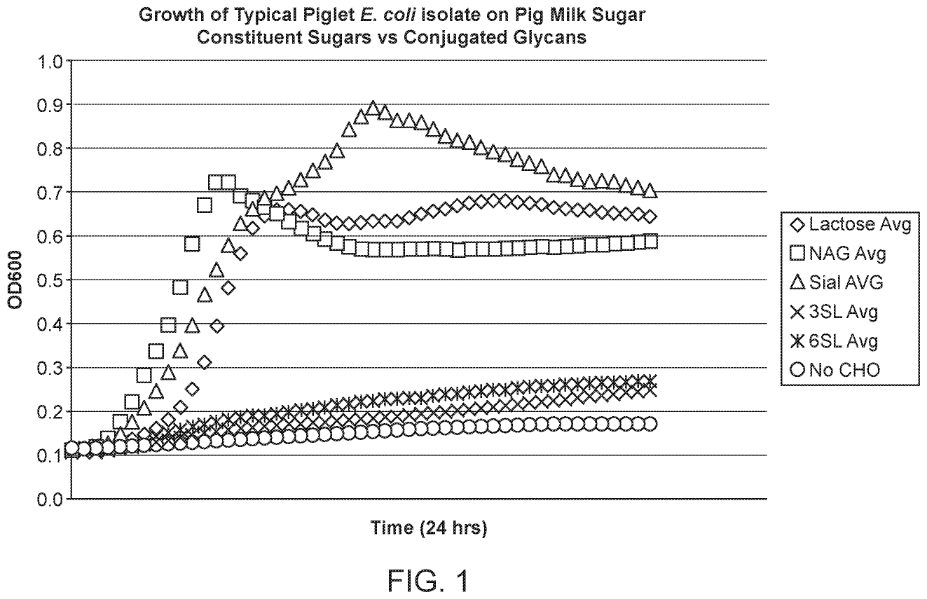

Published Bacteroides genomes contain sequences encoding sialidase enzymes, which may separate the sialic acid moiety from sialyllactose, and create an opportunity for E. coli to thrive in the gut of the nursing animal, where it may not be able to thrive without the activity of this enzyme. Similarly, the activity of beta hexosaminidases, which remove N-acetylglucosamine monomers from complex glycans also generate a niche for E. coli in this manner, as piglet-isolated E. coli were found by to also consume N-acetylglucosamine (FIG. 1). To confirm the presence of these enzymes in the animals, genomic microbial DNA was subjected to metagenomic sequencing, to determine the ecosystem's total metabolic capabilities, and assign taxonomic identities to key metabolic roles. Specifically, the release of sialic acid and N-acetylglucosamine from pig dietary glycans was demonstrated to be driven by populations of the gut microbiota.

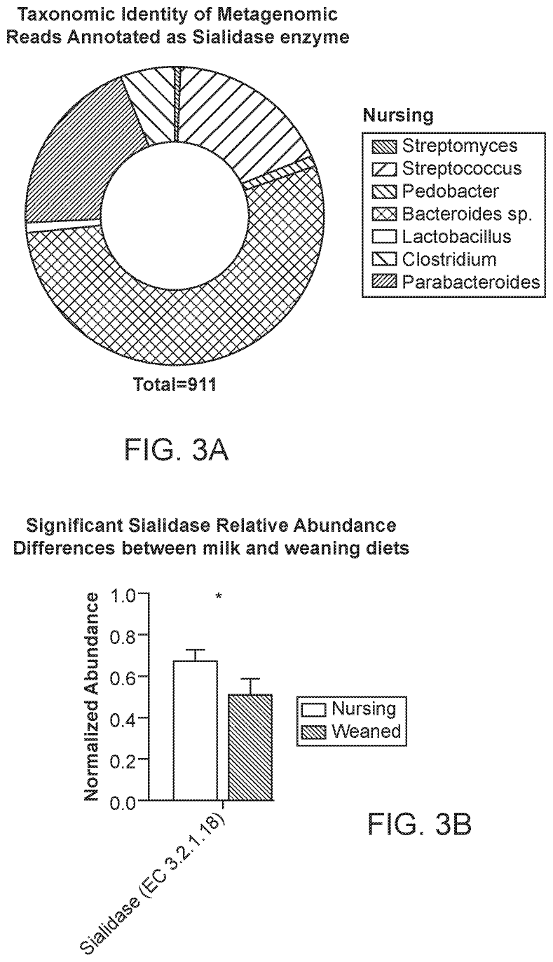

Genes encoding sialidases and beta hexosaminidases were found to belong to members of the gut microbiota. The taxonomic identity of the bacteria housing these specific enzymes were found to be mostly Bacteroides associated with the nursing pigs which diminished when the pigs were weaned (FIG. 3A). Further, the overall abundance of sequencing reads that could be mapped to sialidases declined when the diet of the animals changed to one which contained less of these sugars, suggesting that this enzyme is functionally relevant to populations associated with the pig milk diet but not with the weaned diet composed primarily of oats (FIG. 3B).

Reads that could be classified as a sialidase enzyme and identified taxonomically within the Bacteroides were assembled using velvet, to create a full-length hypothetical sialidase sequence. One of the contigs from this assembly was found to contain a full length sialidase-encoding gene belonging to Bacteroides fragilis, and matched this gene sequence at 99% nucleotide identity, and was used to generate primers that would amplify this sequence from the total fecal DNA sample.

PCR amplification of the gene. Primers matching the hypothetical sialidase were constructed. These primers successfully amplified a sequence from the total fecal DNA, which was subsequently sequenced. The verified sequence matched the hypothetical sequence generated from metagenomic reads at 100%.

In parallel, a representative Bacteroides strain was isolated from fecal samples of nursing pigs by isolation on Bacteroides Bile Esculin agar, a selective and discriminative medium for the isolation of Bacteroides. Isolated Bacteroides strains were found to contain the sialidase by PCR, using the same primers designed previously, and verified by subsequent DNA sequencing. The growth of Bacteroides on sialyllactose was observed, as this organism clearly possesses a functional sialidase enzyme (data not shown).

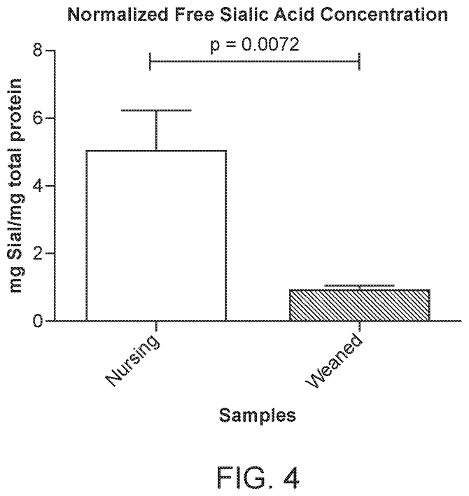

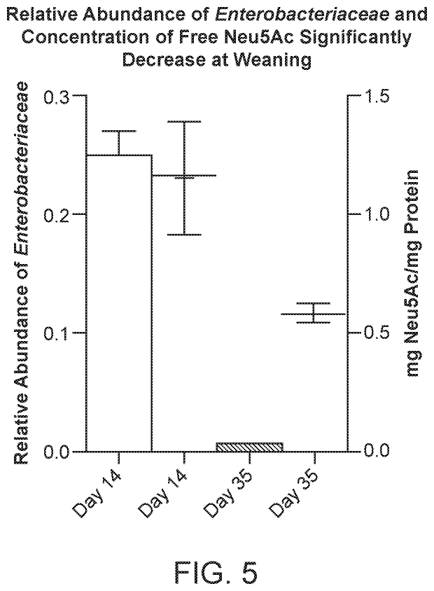

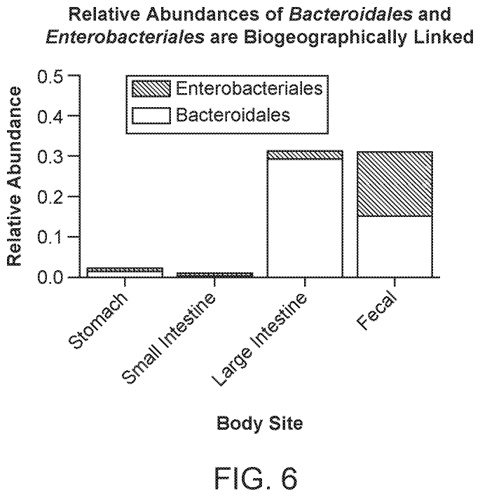

Further, sialic acid concentrations in these fecal samples were compared between nursing and weaning diets and were found to be significantly greater in samples with greater Bacteroides (and thus sialidase enzyme) abundance (FIG. 4). FIG. 5 shows this data from another perspective. On days where there is a high relative abundance of Enterobacteriaceae, there is a high sialic acid concentration in the feces. On days with low Enterobacteriaceae, there is a low concentration of sialic acid. FIG. 5 shows Average Enterobacteriaceae populations over time in pigs (left axis, bars, nursing, blue; weaned, red), are significantly different (p<0.001) between diets as well as concentrations of free sialic acid, p<0.001 (Right axis, whiskers, nursing, blue; weaned, red). FIG. 6 shows that this effect appears mostly confined to the caecum and colon of the piglet. There are high Bacteroides in the large intestine but an equal bloom of Enterobacteriaceae in the ensuing feces, suggesting that Bacteroides is indeed creating a substrate (i.e. sialic acid and more) for Enterobacteriaceae to consume. FIG. 6 shows biogeographical relative abundances of Bacteroidales and Enterobacteriaes in the gut of 14 day old nursing pigs.

Thus, the data can be summarized as: (a) that populations of Enterobacteriaceae in the gut of nursing pigs was found to correlate with the abundance of Bacteroides (r2=0.661, p<0.001), (b) and that these populations of Enterobacteriaceae cannot, by themselves, consume sialylated pig milk oligosaccharides, but (c) Bacteroides possess enzymes capable of releasing sialic acid from pig milk oligosaccharides, which is (d) associated with increased abundances of sialic acid in feces, which (e) these Enterobacteriaceae can consume.

The synthesis of this knowledge is that FSMs released from pig dietary glycans leads to increased populations of Enterobacteriaceae in the gut of nursing pigs, creating an environment where the etiological agents of scour can thrive. Specifically, by reducing the abundance of mono-, di-, or oligomeric sugars, which may include glucose, galactose, N-acetylglucosamine, sialic acid, or fucose derived from dietary glycans, populations of Enterobacteriaceae and other potentially pathogenic organisms capable of consuming these glycans, their breakdown products, or monosaccharides and scour will be prevented or reduced in severity.

This could be accomplished by any approach which reduces concentrations of these monomers or glycans composed of these monomers in the gut. For example, introducing a probiotic microorganism which constitutively and competitively consumes these freed components or glycans could be introduced.

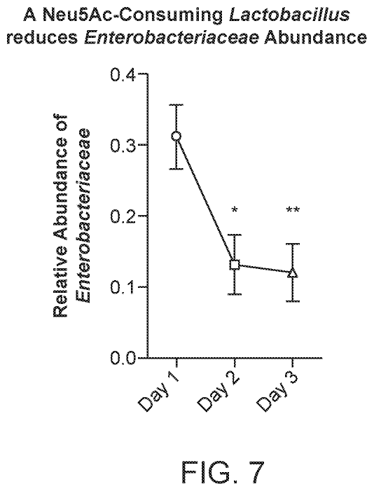

A Lactobacillus reuteri strain was isolated from pig feces that is able to grow on gluconate. This strain was grown to high cell densities and 10¹⁰ CFU was used to gavage 14 d old piglets daily for three days in a pilot experiment. Fecal samples were collected prior to gavage and two days thereafter, and were analyzed by 16S rRNA amplicon sequencing. Importantly, relative populations of Enterobacteriaceae decreased significantly, compared to baseline samples (FIG. 7), despite these populations remaining otherwise stable during nursing in previous studies in age-matched pigs (FIG. 2). Thus, the administration of the Lactobacillus reuteri was effective in reducing Proteobacteria populations. Specifically, FIG. 7 shows the treating 14 d old pigs by gavage with Lactobacillus UCD14261 led to significant reductions in Enterobacteriaceae populations.

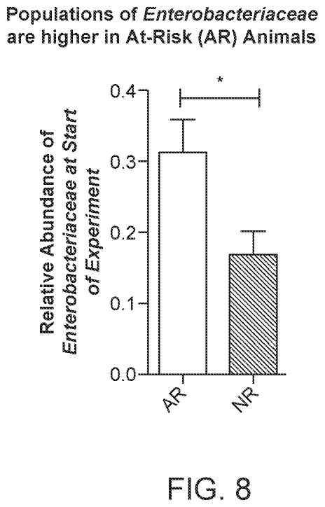

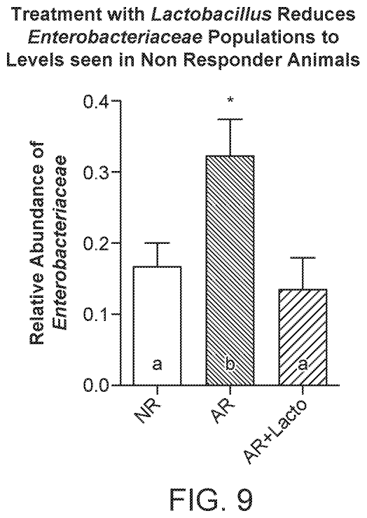

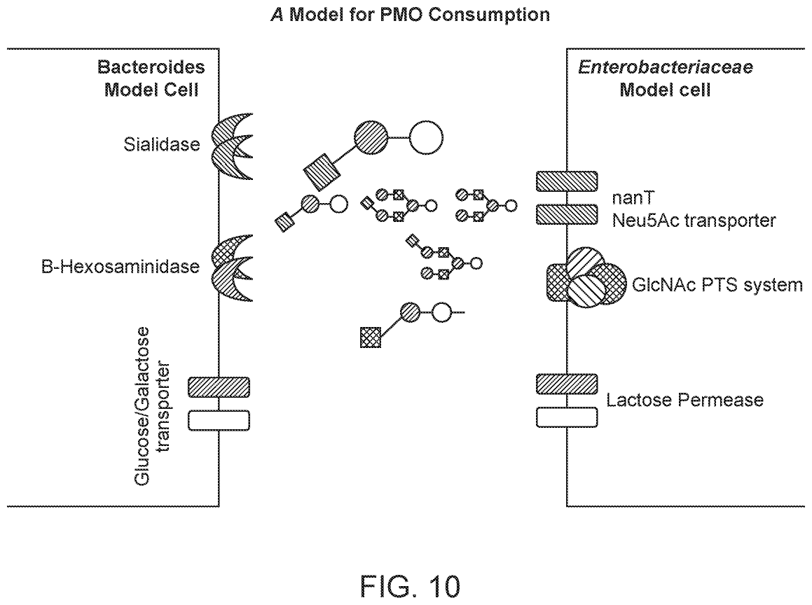

A distinction between populations of piglets was identified even within the same litter. Some animals (7/11) harbored higher (p<0.05) populations of Enterobacteriaceae, which were, on average twice the average population found in low-Enterobacteriaceae animals (4/11 animals) (FIG. 8). FIG. 8 gives distinct differences in “At risk” or high-Enterobacteriaceae versus “NR” or “No risk” pigs prior to gavage with Lactobacillus. These piglets responded differently to supplemented Lactobacillus reuteri UCD14261, where animals harboring high Enterobacteriaceae populations (which were termed “At-Risk” (AR) animals) showed significant drops in these organisms after gavage with Lactobacillus (FIG. 9), populations in the low-Enterobacteriaceae animals were largely unaffected. These “at risk” animals had significantly lower populations of starting Lactobacillaceae populations (p<0.05), which may help explain why higher populations of Enterobacteriaceae could thrive, and why supplementation with Lactobacillus led to a reduction where populations of Enterobacteriaceae were not significantly different from low-Enterobacteriaceae animals. FIG. 9 shows “At risk” (AR) or high-Enterobacteriaceae pigs could be rescued by gavage with Lactobacillus to resemble No Risk or Non Responder animals. Letters denote significance groups (a, b; p<0.05). FIG. 10 shows a model for PMO consumption.

All experiments involving animals were reviewed and approved by the University of California Davis Institutional Animal Care and Use Committee prior to experimentation (Approval #17776, #18279). Throughout the study, all animals were housed in a controlled-access specific pathogen free facility at the University of California Davis dedicated to the rearing of pigs. Three healthy adult pregnant sows from the University of California herd were selected for this study. Upon delivery, the infant pigs were cohoused with sows and ear tagged for identification, following standard practices. The piglets were allowed to nurse freely until weaning after 21 days of age. Piglets were removed from the sow and transferred to separate housing and fed a standard starter feed (Hubbard Feeds Mankato, Minn. USA) after 21 days of age. Animals were given ad libitum access to water and feed. Milk was collected from sows while nursing their respective litters and stored at −80 C.

Fecal samples were collected using a sterile cotton swab (Puritan Medical, Guilford, Me. USA) rectally from each piglet after 1, 3, 5, 7, 14, 21, 28, 35, and 42 days after birth. Swabs were also used to collect fecal samples from mother sows and ^{˜}4 cm2 sites within the enclosure throughout the study.

Sequencing Library Construction. DNA was extracted from swabs using the Zymo Research Fecal DNA kit (Zymo Research Irvine, Calif. USA) according to the manufacturer's instructions. Extracted DNA was used as a template for PCR using barcoded primers to amplify the V4 region of the 16S rRNA gene as previously described for bacteria and the internal transcribed spacer region (ITS) to assess fungal communities.

Briefly, the V4 domain of the 16S rRNA gene was amplified using primers F515 (5′-NNNNNNNNGTGTGCCAGCMGCCGCGGTAA-3′) and R806 (5′-GGACTACHVGGGTWTCTAAT-3′), where the poly-N(italicized) sequence was an 8-nt barcode unique to each sample and a 2-nt linker sequence (bold). PCR amplification was carried out in a 15 μL reaction containing 1× GoTaq Green Mastermix (Promega, Madison, Wis. USA), 1 mM MgCl2, and 2 pmol of each primer. The amplification conditions included an initial denaturation step of 2 minutes at 94° C., followed by 25 cycles of 94° C. for 45 seconds, 50° C. for 60 seconds, and 72° C. for 90 seconds, followed by a single final extension step at 72° C. for 10 minutes. All primers used in this study are summarized in Table S1. Amplicons were pooled and purified using a Qiagen PCR purification column (Qiagen) and submitted to the UC Davis Genome Center DNA Technologies Sequencing Core for paired-end library preparation, cluster generation and 250 bp paired-end sequencing on an Illumina MiSeq. Fungal and bacterial amplicons were sequenced in separate MiSeq runsQuality-filtered demultiplexed reads were analyzed using QIIME 1.8.0 as previously described, except the 13_8 greengenes database release was used for OTU picking and taxonomy assignment and bacterial sequences were aligned using UCLUST. 7 000 sequences per sample were randomly subsampled for analysis of bacterial communities to ensure suitable comparisons. Samples with fewer than 7 000 sequences were omitted. Alpha diversity estimates were computed for phylogenetic diversity (PD) whole tree and compared by nonparametric two-sample t-test with Bonferroni correction and 999 Monte Carlo permutations for bacterial analyses. Beta diversity was calculated by weighted (or unweighted, where noted) UNIFRAC metrics for bacterial populations.

Metagenome sequencing. Total genomic DNA was extracted from fecal samples with the ZYMO Research Fecal DNA Extraction kit according to manufacturer instructions and prepared using the Illumina MiSeq v3 Reagent Chemistry for whole genome shotgun sequencing of multiplexed 150 bp libraries at the University of California Davis Genome Sequencing Core (available on the world wide web at dnatech.genomecenter.ucdavis.edu). Samples were pooled and sequenced across triplicate sequencing runs. FASTQ files were demultiplexed, quality filtered, trimmed to 150 bp, and then reads for each sample were pooled from the three runs, yielding 15-20 million reads per sample, and submitted to the MGRAST pipeline for analysis, which removes host genomic DNA reads and duplicate reads, bins 16S rRNA reads, and functionally classifies remaining reads by predicted protein sequence. Classified reads were normalized in MGRAST and compared between treatments using STAMP.

Isolation of PMG-Consuming Bacteroides and Escherichia coli. Fecal samples were diluted in phosphate buffered saline (pH 7.0) and plated onto pre-reduced Bacteroides Bile Esculin Agar (HiMedia Mumbai, India) plates and incubated at 37° C. anaerobically for 2 d, then subcultured to purity and typed using a MALDI-TOF Biotyper (Bruker Corporation Fremont Calif., USA) according to manufacturer's instructions. 16S rRNA sequencing using primers 8 F and 1391R were used to confirm identity. Bacteroides were cultured in BHI-S overnight, anerobically at 37° C. Bacteroides was grown in minimal medium for growth assays, as described previously, using lactose, glucose, galactose, 2,3-sialyllactose, 2,6-sialyllactose, sialic acid as sole carbon sources (1% w/v).

Identification of sialic-acid consuming Lactobacillus species. Fecal samples from nursing and weaned pigs were cultured on Rogosa SL media containing glucose, raffinose, or ribose as sole carbon sources and grown at 37 or 45 C anaerobically, to preferentially isolate species of Lactobacillus. Colonies were isolated to purity and initially identified using a MALDI-TOF Mass Spectrometer and BioTyper system (Bruker, Fremont, Calif. USA). Genomic DNA was extracted as described previously and partial 16S rRNA sequences were generated by PCR using primers 8 F and 581R under cycling and reaction conditions described elsewhere. Isolates were grouped at the species level and representatives selected for growth screening and 16S rRNA determination. Sequences were determined by the UC Davis DNA Sequencing Core (http://dnaseq.ucdaysis.edu) and compared to the NCBI 16S rRNA database to confirm MALDI-BioTyper identification. Representative isolates were screened for the ability to grow on (1% w/v) sialic acid or N-acetylglucosamine as sole carbon sources in basal MRS medium containing these as a sole carbon source. Lactose and glucose were also compared as positive controls. Lactobacillus genomes available in the JGI-IMG database were screened for the presence of a complete sialic acid utilization repertoire.

Genome Sequencing. Lactobacilli, Bacteroides spp. isolated from nursing piglet fecal samples and possessing the sialidase predicted by metagenomic sequencing, and the Escherichia coli containing the sialic acid catabolism pathway as determined by PCR, were selected for whole genome shotgun sequencing on an Illumina HiSeq at the UC Berkeley Vincent J. Coates Genomics Sequencing Laboratory (found on the world wide web at qb3.berkeley.edu/qb3/gsl/index.cfm). Reads were assembled using velvet, yielding an average coverage >20-fold, and uploaded to the JGI database for annotation and public deposition.

Detection of sialic acid in feces. Fecal samples were suspended in 500 uL of dH2O and vortexted for 30 m at 2500 RPM and then centrifuged at 14 000 RPM for 15 minutes, from which the supernatant was removed. Two additional extractions of the pellet were performed for a final volume of 1.5 mL. 150 uL was removed for protein quantification using the Bradford Assay, with BSA to generate a standard curve. Samples were purified on an anion-exchange resin and eluted with 5 mL 50 mM NaCl and dried under vacuum before reconstituting in 500 uL dH2O. Sialic acid concentrations were determined using a commercial kit according to the manufacturer's instructions (Abcam Cambridge, Mass. USA). The sialic acid concentration was normalized to total protein concentration and expressed as mg sialic acid per mg protein.

Statistical Analysis. T-tests and linear correlations were calculated using Graph Pad Prism 6 for OSX (Graph Pad Software, La Jolla, Calif. USA) with a minimum p value of 0.05.

1.-60. (canceled)

61. A composition comprising at least two non-pathogenic microbes, wherein one of the at least two non-pathogenic microbes is from a first species capable of internalizing and/or metabolizing dietary glycans, and wherein one of the at least two non-pathogenic microbes is from a second species capable of consuming and metabolizing free sugar monomers.

62. The composition of claim 61, wherein the composition is capable of growing on dietary glycans,
wherein the dietary glycans comprises sialic acid and/or fucose, and from 1-10% of sialic acid and/or from 1-10% of the fucose, or from less than 1% of sialic acid and less than 1% of the fucose remains as free sugar monomers after a culture of the composition has ceased to grow; and
wherein at least one of the non-pathogenic microbes comprises comprises a gene coding for
a sialidase or a fucosidase, which may be in the first species of the non-pathogenic microbe; or
a sialic acid or a fucose transporter, which may be in the first species of the non-pathogenic microbe; or
a complex oligosaccharide transporter.

63. The composition of claim 61, wherein the first species of non-pathogenic microbe is selected from the group consisting of
a member of the genus Bifidobacterium;
a member of the genus Bifidobacterium, wherein the Bifidobacterium is B. longum, B. breve, or B. pseudocatenulaturn;
a member of the genus Bifidobacterium, wherein the Bifidobacterium is B. longum subsp. infantis; and
B. longum subsp. infantis, wherein the B. longum subsp. infantis is activated.

64. The composition of claim 61,
wherein the free sugar monomers include fucose, sialic acid, N-acetylglucosamine, N-acetylgalactosamine, gluconate, glucose, galactose, glucosinate, lactose, sialyllactose, fucosyllactose, lacto-N-biose, and mixtures thereof; or
wherein the second species is selected based on the free sugar monomers that are present or predicted to be present in an infant mammal; or
wherein the second species is selected based on the free sugar monomers that are present in an infant mammal, the presence being determined by measuring the free sugar monomers in a fecal sample of the infant mammal; or
wherein the second species is selected based on the free sugar monomers that are present in an infant mammal, the presence being determined by measuring the free sugar monomers in a fecal sample of the infant mammal in an amount of at least 5 μg of free sugar monomer per gram of dry weight of feces; or
wherein the free sugar monomers are predicted to be present in an infant mammal, and wherein the presence is predicted based on a pathogenic bloom of the infant mammal; or
wherein the free sugar monomers are predicted to be present in an infant mammal based on a pathogenic bloom of the infant mammal, the pathogen of the pathogenic bloom optionally being a member of the Class Clostridium or Phylum Proteobacteria; or
wherein the free sugar monomers are predicted to be present in an infant mammal based on a pathogenic bloom of the infant mammal, the pathogen of the pathogenic bloom optionally being C. difficile or C. perfringens; or
wherein the second species is selected based on the free sugar monomers that are preferred by a pathogen, optionally wherein there is an increased likelihood of a pathogenic overpopulation of the pathogen in an infant mammal; or
wherein the second species is selected based on the free sugar monomers that are preferred by a pathogen, optionally wherein there is an increased likelihood of a pathogenic overpopulation of the pathogen in an infant mammal due to an outbreak in the surroundings of the infant mammal; or
wherein the second species is selected based on the free sugar monomers that are preferred by a pathogen, wherein there is an increased likelihood of a pathogenic overpopulation of the pathogen in an infant mammal, and wherein the pathogen is a member of the Class Clostridia or Phylum Proteobacteria; or
wherein the second species is selected based on the free sugar monomers that are preferred by a pathogen, wherein there is an increased likelihood of a pathogenic overpopulation of the pathogen in an infant mammal, and wherein the pathogen is C. difficile or C. perfringens.

65. The composition of claim 61, wherein the second species of non-pathogenic microbe is
a member of the genus Bifidobacterium, Lactobacillus, and/or Pediococcus; or
B. infantis, B. breve, B. bifidum, B. longum, B. adolescentis, B. animalis, or B. pseudocatenulatum; or
L. planatarum, L. casei, L. rhamnosus, L. brevis, L. fermenturn, L. crispatus, L. johnsonii, L. gasseri, L. mucosae, or L. salivarius; or
P. acidilacti, P. entosaceus, P. stilesii, P. argentinicus, or P. claussenii.

66. The composition of claim 61, wherein
the non-pathogenic microbes are present in an amount of 10 million to 500 billion cfu per gram; or
the first species of non-pathogenic microbe is present in an amount of about 10% to about 90% of total amount of the non-pathogenic microbes; or
the first species of non-pathogenic microbe is present in an amount of about 20% to about 80% of total amount of the non-pathogenic microbes; or
the second species of non-pathogenic microbe is present in an amount of about 10% to about 90% of total amount of non-pathogenic microbes; or
the second species of non-pathogenic microbe is present in an amount of about 20% to about 80% of total amount of non-pathogenic microbes; or
the first species is present in an amount of between 10⁴ cfu and 10¹² cfu per gram dry weight; or
the second species is present in an amount of between 10⁴ cfu and 10¹² cfu per gram dry weight.

67. The composition of claim 61, wherein
the composition is in the form of a dry powder or a dry powder suspended in an oil; or
the composition is spray dried or freeze-dried; or
the composition is freeze-dried in the presence of a suitable cryoprotectant;
and if the composition is freeze-dried in the presence of a suitable cryoprotectant, optionally the suitable cryoprotectant is glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, or taurine.

68. The composition of claim 61, further comprising
about 5 to 90% by weight dietary glycans: or
about 5 to 90% by weight dietary glycans derived from a human, swine, or bovine source.

69. A method of improving the health of or treating dysbiosis in a subject, the method comprising
administering to subject, the composition of claim 61; or
administering, based on the presence of said free sugar monomers in the subject's feces, a composition comprising non-pathogenic microbes, wherein the non-pathogenic microbes are capable of metabolizing and/or sequestering the free sugar monomers; or
administering, based on the presence of said free sugar monomers in the subject's feces, a composition comprising non-pathogenic microbes, the non-pathogenic microbes being capable of metabolizing and/or sequestering the free sugar monomers, the composition further comprising a non-pathogenic microbe capable of internalizing and/or metabolizing dietary glycans.

70. A method of improving the health or treating dysbiosis in an infant mammal, the method comprising administering to the infant mammal the composition of claim 61, wherein the infant mammal is receiving dietary glycans contemporaneously with the administration of the composition or the infant mammal is a nursing infant mammal.

71. The method of claim 69, wherein the subject is a mammal; optionally a mammal that is a human, a cow, a pig, a rabbit, a goat, a sheep, a cat, a dog, a horse, or a camel; or a mammal that is an infant.

72. The method of claim 69, wherein the subject is an infant mammal to whom the composition of claim 61 has been administered,
wherein the free sugar monomers consumed by the second species are those present as a consequence of prior antibiotic administration; or
wherein the composition is first administered at a period of within 96 hours of the birth of the infant mammal.

73. A method of detecting a dysbiotic subject, comprising determining a presence of free sugar monomers in the subject's feces and reporting the presence of free sugar monomers.


SEQUENCE LISTING (2 sequences)

SEQ ID NO: 1 - DNA, 29 bp, Artificial Sequence
  F515 Primer
  misc_feature (1)..(8): n is a, c, g, or t
  misc_feature (19)..(19): M can be a or c
nnnnnnnngtgtgccagcmgccgcggtaa

SEQ ID NO: 2 - DNA, 20 bp, Artificial Sequence
  R806 Primer
  misc_feature (8)..(8): h can be a, c, or t/u
  misc_feature (9)..(9): v can be a, g, or c
  misc_feature (14)..(14): w can be a or t/u
ggactachvgggtwtctaat